(19) United States
(12) Patent Application Publication
(10) Pub. No.: US 2014/0187970 A1
(43) Pub. Date: Jul. 3, 2014

(54) OPTICAL PROBE APPARATUS, SYSTEMS, METHODS FOR GUIDING TISSUE ASESSMENT

(71) Applicants: MELISSA J. SUTER, Charlestown, MA (US); Lida P. Hariri, Boston, MA (US); Khay M. Tan, Eunos Crescent (SG); Chunmin Chee, Alberta, CA (US); Milen Shiskhov, Watertown, MA (US); Brett Eugene Bouma, Quincy, MA (US)

(72) Inventors: MELISSA J. SUTER, Charlestown, MA (US); Lida P. Hariri, Boston, MA (US); Khay M. Tan, Eunos Crescent (SG); Chunmin Chee, Alberta, CA (US); Milen Shiskhov, Watertown, MA (US); Brett Eugene Bouma, Quincy, MA (US)

(73) Assignee: The General Hospital Corporation, Boston, MA (US)

(21) Appl. No.: 14/142,995

(22) Filed: Dec. 30, 2013

Related U.S. Application Data
(60) Provisional application No. 61/746,752, filed on Dec. 28, 2012.
(60) Provisional application No. 61/799,109, filed on Mar. 15, 2013.

(51) Int. Cl. A61B 5/00

(52) U.S. Cl. CPC A61B 5/0084; A61B 5/6848
USPC 600/478

(57) ABSTRACT

An exemplary system can be provided for obtaining information associated with at least one tissue. The exemplary system can include at least one waveguide first arrangement which can provide at least one first radiation to the tissue(s), and which can receive at least one optical second radiation from the at least one tissue. Further, at least one configuration can be provided that can transceive at least one electrical signal to and from at least one portion of the system. In addition, at least one computing second arrangement can be provided which may configured to obtain the information based on the second radiation and data corresponding to the electrical signal(s). The data can comprise a position of the portion(s).

### CROSS-REFERENCE TO RELATED APPLICATION(S)

This application relates to U.S. Patent Application Ser. No. 61/746,752 filed Dec. 28, 2012, and U.S. Patent Application Ser. No. 61/799,109 filed Mar. 15, 2013, the entire disclosures of which are incorporated herein by reference.

### STATEMENT OF FEDERAL SUPPORT

The present disclosure was made, at least in part, with support under a Biomedical Research Grant award number RG-194681 from the American Lung Association. Thus, the American Lung Association has certain rights to the disclosure described and claimed herein.

### FIELD OF THE DISCLOSURE

The present disclosure relates to an apparatus for tissue assessment and navigation, and more particularly to exemplary embodiments of a flexible smart optical imaging probe, a smart needle, and smart biopsy forceps, and methods for using the same.

### BACKGROUND INFORMATION

Diagnosis of malignancy and other diseases of the gastrointestinal tract, the pulmonary tract and lung, and many other internal organs are typically made by excising tissue specimens for assessment, which may be accomplished by forceps or core needle biopsy, or by needle aspiration. These techniques for retrieving tissue samples for assessment and diagnosis are often associated with unacceptably low diagnostic yields as a result of incorrect guiding of the forceps or needle to the target tissue, inadvertent biopsy of adjacent normal tissue, and/or biopsy of non-diagnostic tissues within the target region. A use of a smart needle or forceps that provides guidance to the targeted tissue region of interest such as a pulmonary lesion, and confirmation of the needle or forceps placement within the lesion of interest prior to tissue collection can result in an increase in the diagnostic yield.

For example, lung cancer is the leading cause of cancer related death [see Refs. 1-3]. It is well established that early detection and diagnosis greatly increases patient survival [see Ref. 4]. Macroscopic imaging techniques such as computed tomography (CT) can be highly sensitive at detecting small, ≦2 cm, peripheral pulmonary lesions in the lung but they lack the specificity necessary for diagnosis. The diagnostic yield of low-risk bronchoscopy based techniques such as transbronchial needle aspiration can be as low as 14-33% for nodules <2 cm in diameter even with the acquisition of 4-8 serial tissue specimens. (see Refs. 5-8) Even when the biopsy procedure is performed in conjunction with fluoroscopy, endobronchial ultrasound, CT, electromagnetic (EM) navigation or a combination of procedures the associated biopsy yield is still unacceptably low (ranges reported from 44% to 80%). (See Refs.9-13) As a result many patients are advised to undergo a follow-up CT imaging procedure, rather than a definitive diagnostic test, to observe potential growth of the CT detected nodule over time as an indicator of the likelihood of cancer. (See Refs. 14, 15) This is the well-known lung cancer paradox that, while early detection is possible, subsequent diagnosis of these small nodules remains problematic. Accurate and early diagnosis of lung cancer is often hampered by the low diagnostic yield of bronchial biopsy, due to insufficiently large, and inappropriately located tissue sampling.

EM navigation systems have been increasingly used to guide low-risk transbronchial biopsy acquisition and have resulted in reported increases in diagnostic yields, e.g., up to 67%. (See Ref 16) EM navigation relies on tracking a small sensor that is typically attached to guide sheath, within a low frequency electromagnetic field that is generated by a board that is placed beneath the patient. Typically a high resolution CT is first obtained to generate the virtual environment and target lesions are identified. A number of reference points are identified to ensure accurate registration between the patient and the CT virtual environment. Following this initial registration, the guide sheath can subsequently be advanced to the nodule while observing its relative position on the virtual environment. Upon reaching an airway in close proximity to the nodule, a needle can be advanced through the sheath to acquire a biopsy sample.

Optical Coherence Tomography (“OCT”) is a non-invasive imaging modality/tool/procedure/system that can rapidly generate high-resolution (<10 μm) cross-sectional images of biological tissues with penetration depths approaching 2-3 mm (see Fig. A1). (See Refs. 17, 18). When coupled with appropriate catheter designs OCT can be used to conduct in vivo microscopy of tissue microstructure (See Ref 19) including the detection and diagnosis of cardiovascular (see Refs. 20-22) and gastrointestinal (see Refs. 18, 23, 24) pathology. Recently OCT has also been utilized to investigate the tracheobronchial tree in the clinical setting. (See Ref 25) Notably, OCT has been demonstrated to differentiate dysplasia, carcinoma in situ, and invasive cancer in the airways from normal bronchial mucosa. (See Refs. 25-27).

Due to certain limitations in catheter designs, OCT and other optical assessment modalities have been traditionally restricted to imaging of luminal organs, however the development of needle-based catheters promises to extend the utility of OCT beyond these boundaries. For minimally invasive interstitial imaging of tissues and organs, small diameter rigid OCT needle probes have been developed [see Refs. 28-33]. In these designs, the imaging optics, which typically consist of a ball-lens [see Ref 29] or a fiber gradient-index (GRIN) lens [see Refs. 31-33] design, are housed within the stainless steel hypodermic needles. To facilitate a circumferential scanning, a notch is cut in one side of the needle to form a window, and the entire needle is rotated through 360°.

Certain limitations to the design of these OCT needles can include the inability to obtain tissue specimens for diagnosis through the same imaging needle, the incompatibility with standard endoscopy procedures, and due to the direct contact of the mechanically scanning needle with the tissue, unintentional tissue damage or tissue drag may occur distorting the images acquired. Recently, an OCT-guided core-needle biopsy system incorporating a vacuum-assisted rigid breast needle biopsy console has been described, which was modified to accommodate the OCT imaging probe [see Ref. 34]. This publication highlights the potential of the OCT image guidance for a biopsy site selection, although, at present, is limited to rigid needle designs and therefore is not compatible with transbronchial procedures.

Accordingly, there may be a need to address and/or overcome at least some of the issues of deficiencies described herein above.

### SUMMARY OF EXEMPLARY EMBODIMENTS

It is one of the objects of the present disclosure to reduce or address the deficiencies and/or limitations of such prior art approaches, procedures, methods, systems, apparatus and computer-accessible medium.

It may be beneficial to provide an exemplary electromagnetic navigation of the OCT catheter position during imaging, which can facilitate a generation of accurate 3D datasets to more accurately describe the tissue structure and function.

Further, it may be beneficial to provide an example embodiment according to the present disclosure to improve the unacceptably low diagnostic yield that is associated with low-risk bronchial biopsy. According to certain exemplary embodiments of the present disclosure, a novel high-resolution multimodality biopsy guidance platform, system and method can be provided which can utilize external imaging techniques, such as, but not limited to, e.g., CT and electromagnetic navigation for spatial guidance to the nodule and optical coherence tomography (OCT) for microscopic confirmation that the biopsy tool is correctly positioned within the targeted nodule prior to tissue specimen collection.

OCT and other optical imaging techniques and/or modalities can be used to generate three-dimensional (3D) image datasets to comprehensively describe the tissue microenvironment. One exemplary method can be utilized to rotate and simultaneously translate the catheter to conduct spiral cross-sectional imaging however, without accurate knowledge of the precise path of the imaging catheter these 3D representations may be distorted to pseudo-3D images that do not accurately describe the tissue structure. EM tracking of the OCT catheter will enable us to perform accurate 3D imaging of the tissue microenvironment by simultaneously tracking the tip of the OCT catheter in space during image acquisition.

The use of exemplary embodiments of a smart needle and/or forceps arrangements according to the present disclosure can increase diagnostic yield by a) providing guidance to the targeted tissue region of interest, such as a pulmonary lesion; b) providing true three-dimensional assessments of the tissues within the target region; and c) assessing placement of the exemplary needle and/or forceps arrangement(s) within the lesion of interest prior to tissue collection.

Herein, exemplary embodiments of systems, apparatus, methods, and computer-accessible medium, which can utilize optical techniques and/or electromagnetic navigation techniques for the assessment of tissue structure and function e.g. for biopsy guidance exemplary embodiments of methods, apparatus and computer-accessible medium for providing optical assessment of tissue prior to, or during, tissue acquisition using a flexible optical imaging smart needle or biopsy forceps, e.g., for navigation or volumetric evaluation exemplary embodiments of methods, apparatus and computer-accessible medium for providing catheter position data and optical information for accurate volumetric image reconstruction, and methods for using the same. The optical exemplary technique can comprise optical coherence tomography, optical frequency domain imaging, speckle imaging, refractive index measurement, absorption, autofluorescence, diffuse spectroscopy, and/or photoacoustic procedure(s).

In one exemplary embodiment, the OCT smart needle or biopsy forceps can be designed to facilitate both OCT imaging and subsequent specimen collection for a diagnosis, e.g., without removing or repositioning the needle or forceps. The apparatus may consist of an independently actuated OCT needle to first confirm that the tissue of interest has been accurately targeted and a secondary independently actuated apparatus to collect the tissue. In one exemplary embodiment, this secondary apparatus may consist of grasping forceps. In another exemplary embodiment the secondary apparatus may consist of a needle surrounding the first, and in yet another exemplary embodiment the secondary apparatus may consist of a parallel needle.

In a further exemplary embodiment, the OCT catheter can be designed to include an electromagnetic sensor to facilitate both OCT imaging and simultaneous collection of sensor information to determine the spatial orientation of the catheter. In a particular exemplary embodiment of the present disclosure, the transmission of the electrical signal to and or from the sensor may be conducted through a metallic coating or material surrounding the optical imaging fiber. In another exemplary embodiment electrical conductance may be through the drive shaft encasing the optical fiber(s), and in another exemplary embodiment electrical conductance may be through wires parallel to the optical fiber.

For example, EM navigation of the needle into the nodule, rather than navigation of a guide sheath to an airway adjacent to the nodule, can increase the diagnostic yield of low-risk transbronchial biopsy. Further, e.g., a transbronchial OCT imaging catheter can facilitate a confirmation that the needle is within the nodule prior to tissue specimen collection, which can also facilitate an increase of the diagnostic yield of low-risk transbronchial biopsy.

According to a further exemplary embodiment of the present disclosure, a flexible transbronchial optical frequency domain imaging (TB-OFDI) catheter can be provided that functions as a ‘smart needle’ to confirm the needle placement within the target lesion prior to biopsy. The exemplary TB-OFDI smart needle can include a flexible and removable OFDI catheter (e.g., about 430 μm diameter) that can operate within, e.g., a standard 21-gauge TBNA needle. The exemplary OFDI imaging core can be based on an angle polished ball lens design with a working distance of, e.g., about 160 μm from the catheter sheath and a spot size of, e.g., about 25 μm.

Additionally, an exemplary system can be provided for obtaining information associated with at least one tissue. The exemplary system can include at least one waveguide first arrangement which can provide at least one first radiation to the tissue(s), and which can receive at least one optical second radiation from the at least one tissue. Further, at least one configuration can be provided that can transceive at least one electrical signal to and from at least one portion of the system. In addition, at least one computing second arrangement can be provided which may configured to obtain the information based on the second radiation and data corresponding to the electrical signal(s). The data can comprise a position of the portion(s).

For example, the configuration can comprise an electrically-transmitting coating or material which transceives the electrical signal(s). The coating or the material can at least partially cover the first arrangement. The configuration can further comprise a tube arrangement which is configured to transceiver at least one electrical further signal to and from the portion(s) of the system, whereas the coating or the material and the tube arrangement can be electrically separate from one another. The second arrangement can generate the information further based on the electrical further signal(s).

In yet another exemplary embodiment of the present disclosure, the configuration can further comprise a tube arrangement which can be configured to transceiver at least one electrical further signal to and from the portion(s) of the system. The tube arrangement can comprise a drive shaft arrangement. In addition or alternatively, the configuration can comprise at least one electrically-conducting wire which transceives the electrical signal(s). The electrically-conducting wire(s) can comprise a plurality of wires. At least one third arrangement can also be provided that is configured to translate and/or rotate (i) the first arrangement, and/or (ii) the configuration. The second arrangement can determine the position based on a simultaneous detection of the second radiation(s) and the electrical signal(s) during the rotation and/or the translation. The second arrangement can further determine a spatial orientation of the at least one portion based on the simultaneous detection during the at least one of the rotation or the translation. The second arrangement can generate at least one three-dimensional image of the tissue(s) based on the information. The second arrangement can generate the information further based on at least one third radiation provided from a reference.

According to yet another exemplary embodiment of the present disclosure, a probe apparatus can be provided. The exemplary probe apparatus can include at least one first arrangement which can have at least one portion that physically contacts or penetrates at least one section of a tissue, and which can transceive at least one optical radiation to or from such section(s). The exemplary probe can also include at least one second arrangement which can be configured to remove the section(s) from the tissue.

In operation, the portion(s) cab be translated and/or rotated within or near the tissue. The first arrangement can be a needle, and the second arrangement can be a further needle. The needle and the further needle can be situated in the apparatus in a substantially parallel manner with respect to one another. A control arrangement can be provided which is configured to actuate (i) the needle, (ii) the further needle, and/or (iii) the apparatus by rotating and/or translating the same. The control arrangement can actuate the needle and the further needle independently from one another.

The second arrangement can comprise a grasping arrangement which can be configured to grasp the section(s), and move the tissue out of the apparatus. The grasping arrangement can be provided in a parallel configuration to the first arrangement. The grasping arrangement can at least partially enclose the first arrangement. The first arrangement and/or the second arrangement can include a hydrophilic coating.

According to an exemplary embodiment of the present disclosure, simultaneous collection and assessment of probe spatial orientation and optical signal can be used for accurate volumetric image reconstruction of tissue microstructure.

These and other objects, features and advantages of the present invention will become apparent upon reading the following detailed description of embodiments of the disclosure, and the appended claims.

### BRIEF DESCRIPTION OF THE DRAWING

Further objects, features and advantages of the present disclosure will become apparent from the following detailed description taken in conjunction with the accompanying drawings showing illustrative embodiments of the present disclosure, in which:

FIG. 1(a) is a schematic diagram for a distal-end of an OCT smart needle arrangement according to an exemplary embodiment of the present disclosure;

FIG. 1(b) is a schematic diagram for a distal-end of another OCT smart needle arrangement with a cutting tip according to another exemplary embodiment of the present disclosure;

FIG. 1(c) is a photograph of an exemplary flexible OCT catheter insert configuration of the needle illustrated in FIG. 1(a);

FIG. 2—is a schematic diagram of the exemplary smart needle arrangement illustrated in FIG. 1(b), with a hydrophilic coating or material on a catheter sheath and a needle according to an exemplary embodiment of the present disclosure;

FIGS. 3(a)-3(c) are illustrations of functional diagrams of the exemplary OCT smart needle arrangement in operation according to further exemplary embodiment of the present disclosure;

FIG. 4 (a) is a schematic diagram for a distal-end of an OCT smart needle arrangement according to an exemplary embodiment of the present disclosure, with separate OCT needle and tissue acquisition needle bore.

FIG. 4(b) is a schematic diagram for the distal-end of the OCT smart needle arrangement according to an exemplary embodiment of the present disclosure provided in a probe, with independent dual bore needles;

FIG. 5(a) is a schematic diagram for a distal-end of an OCT smart-forceps arrangement according to an exemplary embodiment of the present disclosure, with independent OCT needle and biopsy forceps;

FIG. 5(b) is a schematic diagram for a distal-end of another OCT smart-forceps arrangement according to a further exemplary embodiment of the present disclosure, with an independently actuating OCT smart needle for imaging and tissue acquisition, and independently actuating biopsy forceps for tissue acquisition.

FIGS. 6(a) and 6(b) are illustrations of exemplary longitudinal OCT images, respectively, of an inflated swine lung parenchyma obtained with the exemplary OCT imaging needle arrangement according to an exemplary embodiment of the present disclosure;

FIG. 7(a) is a schematic diagram for a distal-end of an EM-OCT catheter arrangement according to an exemplary embodiment of the present disclosure;

FIG. 7(b) is a schematic diagram for distal-end of an EM-OCT catheter arrangement according to another exemplary embodiment of the present disclosure, in which a metallic coating or material surrounding the optical fiber is used for electrical conductance;

FIG. 7(c) is a schematic diagram for a distal-end of an EM-OCT catheter arrangement according to still another exemplary embodiment of the present disclosure, which includes an electrically conducting drive shaft; and

FIG. 7(d) is a schematic diagram for a distal-end of an EM-OCT catheter arrangement according to yet another exemplary embodiment of the present disclosure, which has a metallic coating or material surrounding the optical fiber for providing an electrical conductance, and in which a drive shaft is used for electrical conductance.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject disclosure and appended claims.

### DETAILED DESCRIPTION OF EXEMPLARY EMBODIMENTS

### Exemplary OCT Smart Catheter

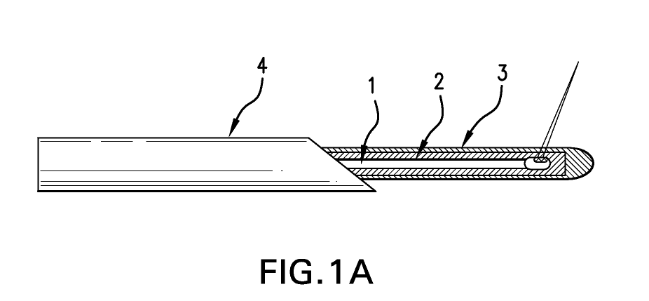

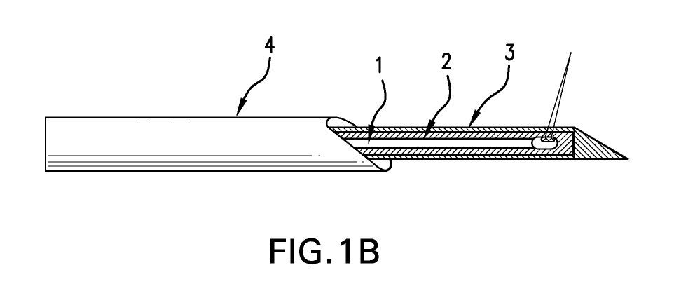

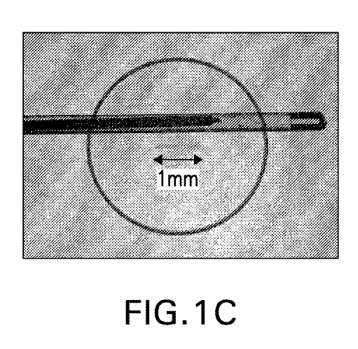

FIG. 1(a) illustrates a schematic diagram for a distal-end of an OCT smart needle arrangement according to an exemplary embodiment of the present disclosure. For example, the illustrated OCT arrangement can be provided as a catheter insert, and can include an imaging core housed inside a transparent polyimide (e.g., catheter) sheath 3. The imaging core consists of an optical fiber imaging probe 1 and a nitinol drive shaft 2. The exemplary catheter insert can slide freely within the needle 4, including, but not limited to, e.g., standard TBNA needles.

FIG. 1(b) is a schematic diagram for a distal-end of an OCT smart needle arrangement with a cutting tip. For example, the cutting tip can be used to aid with a penetration of tissue by the OCT catheter.

FIG. 1(c) illustrates a photograph of an exemplary flexible OCT catheter insert of the needle illustrated in FIG. 1(a);

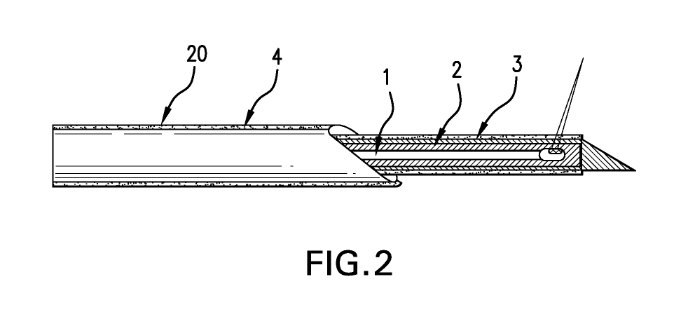

The exemplary OCT smart needle arrangement according to an exemplary embodiment of the present disclosure can come into contact with tissue during imaging. FIG. 2 shows a schematic diagram of the exemplary smart needle arrangement illustrated in FIG. 1(b) with a hydrophilic coating or material 20 provided on the catheter sheath and the needle 4. For example, the use of the hydrophilic coating 20 can reduce friction between the needle 4, catheter sheath 3, and tissue during the tissue penetration and imaging.

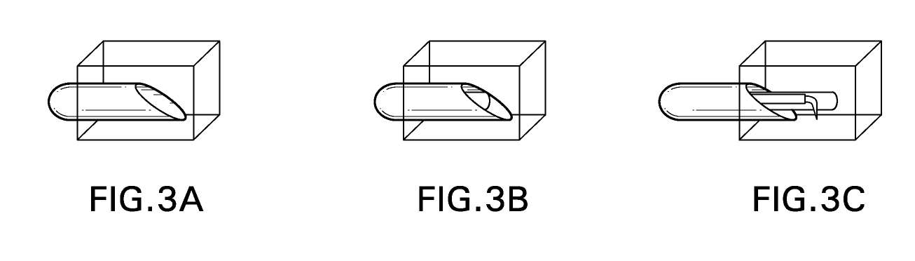

FIGS. 3(a)-3(c) show illustrations of functional diagrams of the exemplary OCT smart needle arrangement(s) in operation, and demonstrate the functionality of the OCT smart needle arrangement. As an initial matter, the exemplary needle can be placed in the tissue(see FIG. 3(a)). The OCT catheter can be advanced within the needle (see FIG. 3(b)). The needle can then be withdrawn, thus exposing the OCT catheter for imaging (see FIG. 3(c)). Exemplary images can be collected and assessed to determine if the needle is placed optimally for tissue biopsy. For example, if the needle is not optimally placed for tissue acquisition, then the OCT catheter can be retracted, and the needle may be repositioned. If the needle is optimally placed, the OCT catheter can be removed from the needle bore, and the tissue biopsy can be collected with the needle.

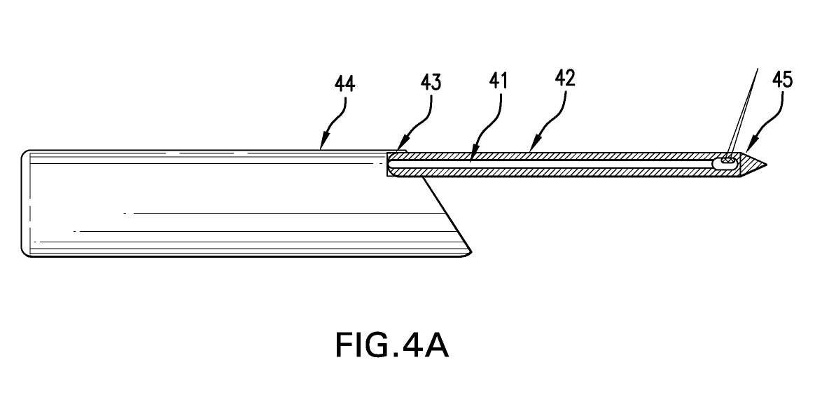

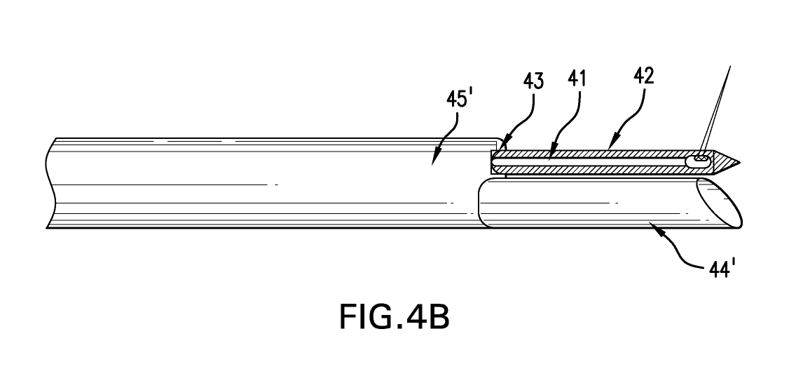

FIG. 4 (a) is a schematic diagram for a distal-end of another OCT smart needle according to another exemplary embodiment of the present disclosure, which has separate OCT (e.g., open bore) needle 45 and tissue acquisition needle bore(s)/channel(s) 43. For example, the needle 44 can be directed toward the tissue of interest. A separate OCT needle 45 can be advanced into the tissues of interest. Images can be collected via an optical fiber 41 (which can be encased by, e.g., metallic coating or hypertube 42) and assessed to determine if the needle 45 is placed optimally for the tissue biopsy. If the needle 45 is not optimally placed for tissue acquisition, then the OCT catheter can be retracted, and the needle 45 is repositioned. If the needle 45 is optimally placed, the OCT catheter is retracted and the larger needle 44 is advanced to collect a tissue biopsy. This exemplary configuration facilitates a collection of the tissue that is provided immediately adjacent to the OCT imaging field, and prevents damage of biopsied tissue by the OCT probe.FIG. 4(b) shows a schematic diagram for a distal-end of an OCT smart needle arrangement according to another exemplary embodiment of the present disclosure which is provided in an encasing probe 45′ with an independent dual bore needle 44′ in addition to another need that is situated parallel thereto. Independent bores (e.g., including the channel/bore 43) have independent actuation capabilities. For example, the exemplary OCT probe arrangement shown in FIG. 4(b) can be actuated and images are collected via the optical fiber 41 to assess the tissue localization. If the exemplary probe is optimally placed to collect tissue, the independent needle can be actuated for tissue collection parallel to the OCT probe. This design facilitates a collection of the tissue situated immediately adjacent to the OCT imaging field, and can reduce or even prevents damage of the biopsied tissue by the exemplary OCT probe.

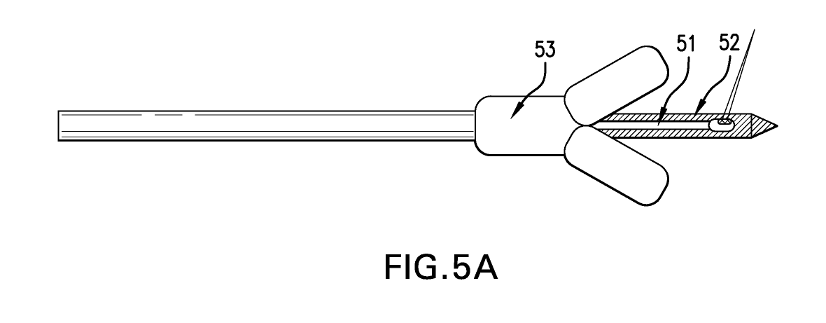

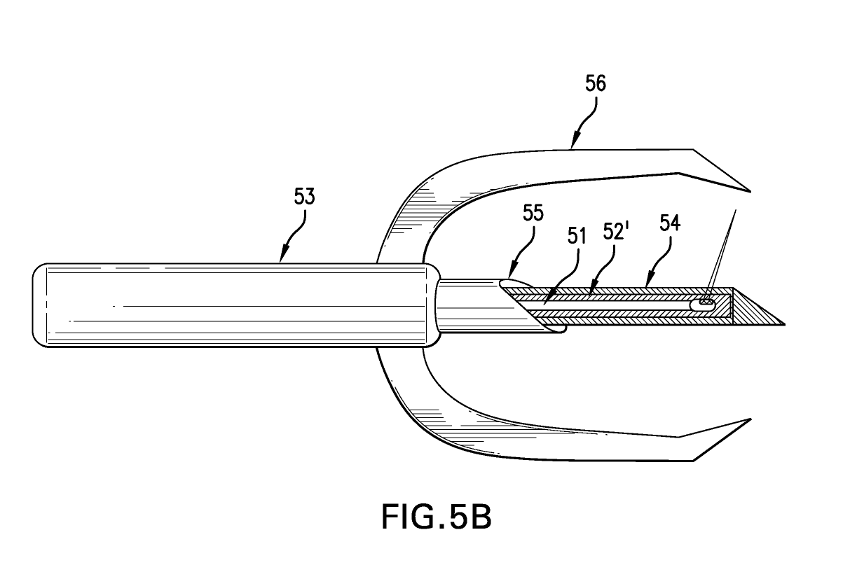

FIG. 5(a) illustrates is a schematic diagram for a distal-end of an OCT smart forceps arrangement according to an exemplary embodiment of the present disclosure, which include an independent OCT needle and biopsy forceps arrangement 53. For example, the forceps arrangement 53 can be placed at or near the region of interest. The exemplary OCT probe can then penetrate the tissue of interest, and OCT images are collected via an optical fiber 51 (which can be encased by, e.g., metallic coating or hypertube 52). If the tissue imaged is satisfactory for biopsy, the exemplary OCT probe can be retracted and a forceps biopsy is collected. If the tissue is not satisfactory for biopsy, the forceps arrangement 53 (or any portion thereof, including individual forceps) can be repositioned, and the procedure is repeated as described above.

FIG. 5(b) shows a schematic diagram for a distal-end of another OCT smart forceps arrangement according to a further exemplary embodiment of the present disclosure. The exemplary arrangement of FIG. 5(b) includes forceps 53, as well as at least one independently actuating OCT smart needle 55 (which can be an open bore needle) for imaging and tissue acquisition, and independently actuating biopsy forceps arrangement 56 for tissue acquisition. In addition, a catheter sheath 54 (enclosing a further needle) can be slidably provided within the OCT smart needle 55. For example, the forceps arrangement 56 (or any portion thereof) can be placed in a region of interest. The exemplary OCT probe can then penetrate the tissue of interest, and OCT images are collected via the optical fiber 51. If the tissue imaged is satisfactory for biopsy, the exemplary OCT probe (including the OCT smart needle 55) can be refracted. This exemplary arrangement can facilitate both needle aspiration and forceps biopsy, and one or both mechanisms can be utilized to obtain tissue.

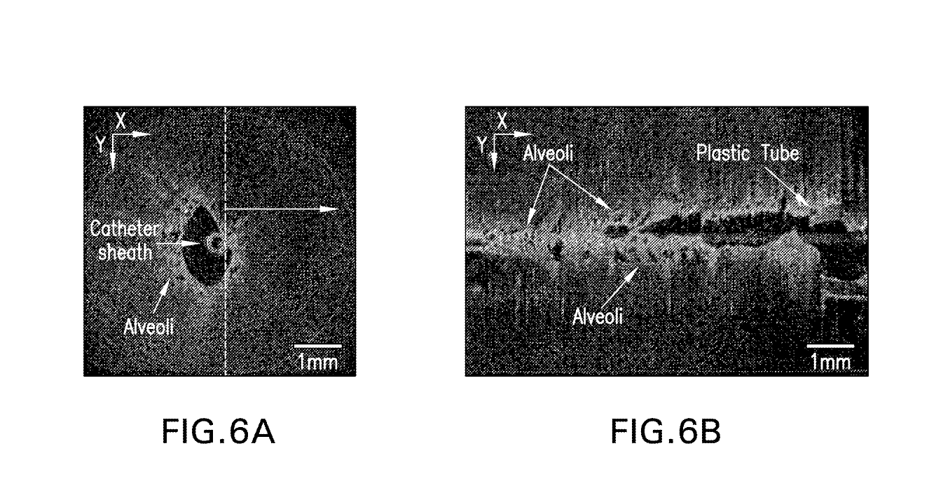

FIGS. 6(a) and 6(b) shows illustrations of exemplary cross-sectional and longitudinal OCT images, respectively, of an inflated swine lung parenchyma obtained with the exemplary OCT imaging needle. Both the cross-sectional images (shown in FIG. 6(a)) and the longitudinal images (shown in FIG. 6(b)) illustrate a clear visualization of alveoli with a fine detail.

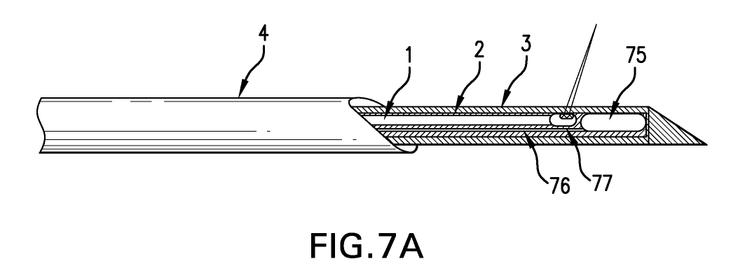

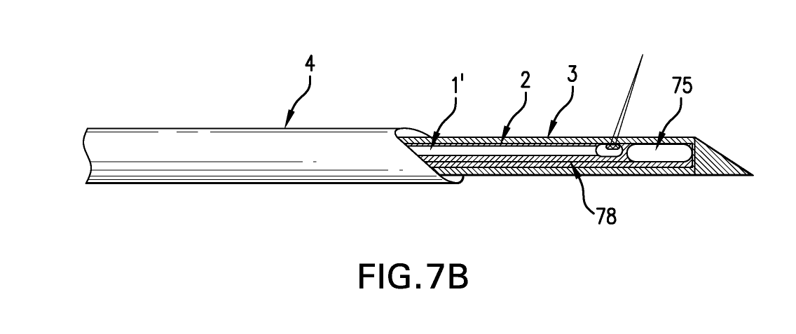

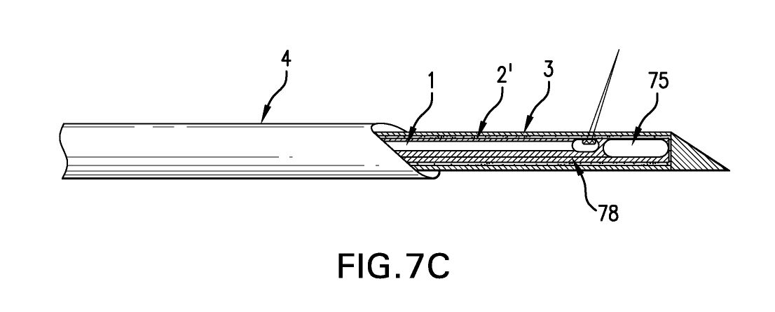

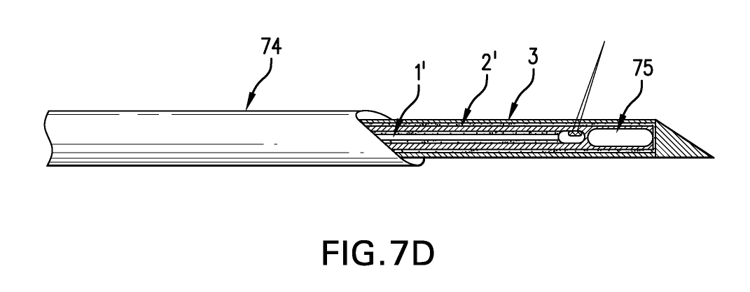

FIG. 7(a) shows a schematic diagram for a distal-end of an EM-OCT catheter arrangement according to an exemplary embodiment of the present disclosure. As shown in FIG. 7(a), a plurality of sensors (e.g., a primary sensor 75, a wire-to sensor 76, and a wire-from sensor 77) are incorporated into the exemplary arrangement. For example, the exemplary OCT arrangement of FIG. 7(a) can be used for positional tracking and guidance to tissues of interest. When the exemplary OCT arrangement has been guided to the tissue of interest with the primary sensor 75 (or with one or both the other sensors 76, 77), the needle 4 can be penetrated into the tissue. The needle 4 can be retracted to expose the OCT catheter. OCT imaging can be collected via the optical fiber 1 which are at or near the adjacent tissues. If the tissue is satisfactory for biopsy, the OCT catheter can be retracted and a needle biopsy/aspiration can be performed.

FIG. 7(b) illustrates a schematic diagram for a distal-end of another EM-OCT catheter arrangement according to an exemplary embodiment of the present disclosure, in which a metallic coating or material surrounding an optical fiber 1′ can be used for the electrical conductance. The exemplary arrangement of FIG. 7(b) can also include a plurality of sensors (e.g., a primary sensor 75, a combined wire-to/wire-from sensor 8). The operation of the exemplary arrangement of FIG. 7(b) is similar to that of FIG. 7(a). FIG. 7(c) shows a schematic diagram for a distal-end of still another EM-OCT catheter according to an exemplary embodiment of the present disclosure, which includes an electrically conducting drive shaft 2′. The operation of the exemplary arrangement of FIG. 7(c) is similar to that of FIG. 7(a). FIG. 7(d) illustrates a schematic diagram for a distal-end of still another EM-OCT catheter arrangement according to yet another exemplary embodiment of the present disclosure, where a metallic coating or material surrounding the optical fiber 1′ can be used for an electrical conductance and a drive shaft 2 is used for electrical conductance. For example, this exemplary arrangement of FIG. 7(d) can utilize the optical fiber 1′ and/or the drive shaft 2′ (which can be the electrically conducting drive shaft) to achieve electrical conductance to and/or from the exemplary sensor 75. The operation of the exemplary arrangement of FIG. 7(d) is similar to that of FIG. 7(b).

For example, the exemplary sensor(s) 75, 76. 77, 78 shown in FIGS. 7(a)-7(d), as applicable can be used to collect and assess information regarding, e.g., a spatial orientation of the exemplary probe, and generate three-dimensional image reconstruction(s) of the tissue microstructure.

### Exemplary OCT Smart Needle Imaging Procedure

To demonstrate the feasibility and image quality of the exemplary OCT smart needle, freshly excised lungs from swine have been imaged. An endotracheal tube was inserted and inflated in the trachea and the lungs were subsequently inflated to a pressure of 20 cmH2O. A bronchoscope (1970K, Pentax, Japan) was then inserted into the endotracheal tube and the airways were examined. Following identification of a target site, the transbronchial needle was inserted into the working channel of the bronchoscope and was maneuvered to puncture the airway wall and enter the parenchyma, as shown in FIG. 3(a). The TBNA stylet was subsequently withdrawn and was replaced with the OCT catheter, as shown in FIG. 3(b). The OCT catheter was locked onto the proximal end of the TBNA needle via a luer lock. When the catheter was advanced to the distal end of the needle, the TBNA needle was withdrawn ˜1-2 cm to expose the OCT catheter for imaging, as shown in FIG. 3(c). Following imaging with the OCT catheter still in place, the TBNA needle was re-advanced over the catheter to the initial position, the OCT catheter was then unlocked and removed from the TBNA needle, and an aspirate or core biopsy obtained for diagnosis. This procedure ensures that the TBNA needle remains within the target tissue for biopsy acquisition following OCT imaging. Imaging of the parenchyma was successfully performed at a number of locations chosen throughout the tracheobronchial tree including the very peripheral regions of the lung. FIGS. 6(a) and 6(b) illustrate exemplary OCT images of the lung parenchyma surrounding the needle in logarithmic gray scale. An exemplary cross-sectional OCT image (see FIG. 6(a)) and a corresponding longitudinal reslice (see FIG. 6(b)) of the volumetric OCT data (e.g., obtained from the position indicated with a light dotted line illustrated in FIG. 6(a)) demonstrates that the OCT smart needle can clearly resolve alveoli (arrows) within the lung parenchyma.

Further Discussion and Exemplary Conclusion

A flexible, narrow diameter OCT smart catheter can be provided that can facilitate an acquisition of high-resolution OCT images of the peripheral lung. The feasibility and usability of the OCT smart needle has been successfully demonstrated on freshly excised inflated swine lungs. The transbronchial imaging procedure was carried out according to standard clinical bronchoscopy transbronchial needle placement procedures. The experimental results suggest that the OCT smart needle may be a useful tool for investigating and potentially increasing the diagnostic yield of peripheral pulmonary lesions.

While the OCT smart needle catheter presented in this manuscript was designed primarily for pulmonary use, the basic design may additionally be useful for other organs systems where needle aspiration or core biopsy procedures are routinely performed.

Although the current catheter sheath is suitably flexible, there is a small risk that it may be damaged or sheared off by the sharp aspiration needle. Further exemplary OCT smart needle designs can include rigid hypodermic tubing into the distal portion of the driveshaft to overcome this issue without the need to dull the needle tip.

The current exemplary OCT imaging needle facilitates, e.g., side-viewing to provide volumetric assessment of the surrounding tissue. A forward-imaging catheter design can be provided to facilitate an assessment of the tissue prior to positioning the needle within the lesion. This may assist with a guide needle placement to the target tissue region of interest and to avoid major blood vessels thereby reducing blood contamination in the OCT images. Forward-imaging catheters have been developed based on the use of coherent fiber bundles [see Ref. 35], paired angled GRIN lenses [see Ref. 36], a GRIN rod [see Ref. 37] and a single-body lensed-fiber design [see Ref. 38]. However, such previously-described catheter designs are difficult to miniaturize while simultaneously providing a sufficiently large imaging field of view.

Using the exemplary OCT imaging needle described herein can provide a beneficial use of, e.g., a smart needle by confirming the placement of the biopsy needle within a peripheral pulmonary lesion prior to biopsy, and can facilitate a real-time optical diagnosis of the lesion. While the OCT smart needle has been described herein for pulmonary use, the exemplary design can also be useful for other organ systems where FNA or core biopsy procedures are routinely performed.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

### EXEMPLARY REFERENCES AND LINKS

1. America Cancer Society, Cancer facts and figures 2010 (ACS, 2010).
2. S. Altekruse, C. Kosary, M. Krapcho, N. Neyman, R. Aminou, W. Waldron, J. Ruhl, N. Howlader, Z. Tatalovich, and H. Cho, “SEER cancer statistics review,” Bethesda, Md.: National Cancer Institute (2010).
3. A. B. Mariotto, K. R. Yabroff, Y. Shao, E. J. Feuer, and M. L. Brown, “Projections of the cost of cancer care in the United States: 2010-2020,” J. Natl. Cancer Inst. 103, 117-128 (2011).
4. W. A. Baaklini, M. A. Reinoso, A. B. Gorin, A. Sharafkaneh, and P. Manian, “Diagnostic yield of fiberoptic bronchoscopy in evaluating solitary pulmonary nodules,” Chest 117, 1049-1054 (2000).
5. Baaklini W A, Reinoso M A, Gorin A B, Sharafkaneh A, Manian P. Diagnostic yield of fiberoptic bronchoscopy in evaluating solitary pulmonary nodules. Chest 2000; 117:1049-1054.
6. Mazzone P, Jain P, Arroliga A C, Matthay R A. Bronchoscopy and needle biopsy techniques for diagnosis and staging of lung cancer. Clin Chest Med 2002; 23:137-158, ix.
7. Shure D, Fedullo P F. Transbronchial needle aspiration of peripheral masses. Am Rev Respir Dis 1983; 128:1090-1092.
8. Dooms C, Seijo L, Gasparini S, Trisolini R, Ninane V, Tournoy KG. Diagnostic bronchoscopy: State of the art. Eur Respir Rev 2010; 19:229-236.
9. Asahina H, Yamazaki K, Onodera Y, Kikuchi E, Shinagawa N, Asano F, Nishimura M. Transbronchial biopsy using endobronchial ultrasonography with a guide sheath and virtual bronchoscopic navigation. Chest 2005; 128:1761-1765.
10. Herth F J, Ernst A, Becker H D. Endobronchial ultrasound-guided transbronchial lung biopsy in solitary pulmonary nodules and peripheral lesions. Eur Respir J 2002; 20:972-974.
11. Eberhardt R, Anantham D, Ernst A, Feller-Kopman D, Herth F. Multimodality bronchoscopic diagnosis of peripheral lung lesions: A randomized controlled trial. Am J Respir Crit Care Med 2007; 176:36-41.
12. Kurimoto N, Miyazawa T, Okimasa S, Maeda A, Oiwa H, Miyazu Y, Murayama M. Endobronchial ultrasonography using a guide sheath increases the ability to diagnose peripheral pulmonary lesions endoscopically. Chest 2004; 126:959-965.
13. Kikuchi E, Yamazaki K, Sukoh N, Kikuchi J, Asahina H, Imura M, Onodera Y, Kurimoto N, Kinoshita I, Nishimura M. Endobronchial ultrasonography with guide-sheath for peripheral pulmonary lesions. Eur Respir J 2004; 24:533-537.
14. Ost D, Fein A M, Feinsilver S H. Clinical practice. The solitary pulmonary nodule. N Engl J Med 2003; 348:2535-2542.
15. Tan B B, Flaherty K R, Kazerooni E A, Iannettoni M D. The solitary pulmonary nodule. Chest 2003; 123:89 S-96S.
16. Eberhardt R, Anantham D, Herth F, Feller-Kopman D, Ernst A. Electromagnetic navigation diagnostic bronchoscopy in peripheral lung lesions. Chest 2007; 131:1800-1805.
17. Huang D, Swanson E A, Lin C P, Schuman J S, Stinson W G, Chang W, Hee M R, Flotte T, Gregory K, Puliafito C A, et al. Optical coherence tomography. Science 1991; 254:1178-1181.
18. Bouma B E, Tearney G J, Compton C C, Nishioka N S. High-resolution imaging of the human esophagus and stomach in vivo using optical coherence tomography. Gastrointest Endosc 2000; 51:467-474.
19. Yun S H, Tearney G J, Vakoc B J, Shishkov M, Oh W Y, Desjardins A E, Suter M J, Chan R C, Evans J A, Jang I K, et al. Comprehensive volumetric optical microscopy in vivo. Nat Med 2006; 12:1429-1433.
20. Brezinski M E, Tearney G J, Bouma B E, Izatt J A, Hee M R, Swanson E A, Southern J F, Fujimoto J G. Optical coherence tomography for optical biopsy. Properties and demonstration of vascular pathology. Circulation 1996; 93:1206-1213.
21. Tearney G J, Waxman S, Shishkov M, Vakoc B J, Suter M J, Freilich M I, Desjardins A E, Oh W Y, Bartlett L A, Rosenberg M, et al. Three-dimensional coronary artery microscopy by intracoronary optical frequency domain imaging. JACC Cardiovasc Imaging 2008; 1:752-761.
22. Fujimoto J G, Boppart S A, Tearney G J, Bouma B E, Pitris C, Brezinski M E. High resolution in vivo intra-arterial imaging with optical coherence tomography. Heart 1999; 82:128-133.
23. Tearney G J, Brezinski M E, Bouma B E, Boppart S A, Pitris C, Southern J F, Fujimoto J G. In vivo endoscopic optical biopsy with optical coherence tomography. Science 1997; 276:2037-2039.
24. Suter M J, Vakoc B J, Yachimski P S, Shishkov M, Lauwers G Y, Mino-Kenudson M, Bouma B E, Nishioka N S, Tearney G J. Comprehensive microscopy of the esophagus in human patients with optical frequency domain imaging. Gastrointest Endosc 2008; 68:745-753.
25. Tsuboi M, Hayashi A, Ikeda N, Honda H, Kato Y, Ichinose S, Kato H. Optical coherence tomography in the diagnosis of bronchial lesions. Lung Cancer 2005; 49:387-394.
26. Lam S, Standish B, Baldwin C, McCaniams A, leRiche J, Gazdar A, Vitkin A I, Yang V, Ikeda N, MacAulay C. In vivo optical coherence tomography imaging of preinvasive bronchial lesions. Clin Cancer Res 2008; 14:2006-2011.
27. Michel R G, Kinasewitz G T, Fung K M, Keddissi J I. Optical coherence tomography as an adjunct to flexible bronchoscopy in the diagnosis of lung cancer: A pilot study. Chest 2010.
28. X. D. Li, C. Chudoba, T. Ko, C. Pitris, and J. G. Fujimoto, “Imaging needle for optical coherence tomography,” Opt. Lett. 25, 1520-1522 (2000).
29. V. X. D. Yang, Y. X. Mao, N. Munce, B. Standish, W. Kucharczyk, N. E. Marcon, B. C. Wilson, and I. A. Vitkin, “Interstitial Doppler optical coherence tomography,” Opt. Lett. 30, 1791-1793 (2005).
30. A. M. Zysk, D. L. Marks, D. Y. Liu, and S. A. Boppart, “Needle-based reflection refractometry of scattering samples using coherence-gated detection,” Opt. Express 15, 4787-4794 (2007).
31. D. Lorenser, X. Yang, R. W. Kirk, B. C. Quirk, R. A. McLaughlin, and D. D. Sampson, “Ultrathin side-viewing needle probe for optical coherence tomography,” Opt. Lett. 36, 3894-3896 (2011).
32. Y. C. Wu, J. F. Xi, L. Huo, J. Padvorac, E. J. Shin, S. A. Giday, A. M. Lennon, M. I. F. Canto, J. H. Hwang, and X. D. Li, “Robust high-resolution fine OCT needle for side-viewing interstitial tissue imaging,” IEEE J. Sel. Top. Quant. 16, 863-869 (2010).
33. B. C. Quirk, R. A. McLaughlin, A. Curatolo, R. W. Kirk, P. B. Noble, and D. D. Sampson, “In situ imaging of lung alveoli with an optical coherence tomography needle probe,” J. Biomed. Opt. 16, 036009 (2011).
34. W. C. Kuo, J. Kim, N. D. Shemonski, E. J. Chaney, D. R. Spillman, and S. A. Boppart, “Real-time three-dimensional optical coherence tomography image-guided core-needle biopsy system,” Biomed. Opt. Express 3, 1149-1161 (2012).
35. J. H. Han, X. Liu, C. G. Song, and J. U. Kang, “Common path optical coherence tomography with fibre bundle probe,” Electron. Lett. 45, 1110-1111 (2009).
36. S. Han, M. V. Sarunic, J. Wu, M. Humayun, and C. H. Yang, “Handheld forward-imaging needle endoscope for ophthalmic optical coherence tomography inspection,” J. Biomed. Opt. 13, 020505 (2008).
37. C. P. Liang, J. Wierwille, T. Moreira, G. Schwartzbauer, M. S. Jafri, C. M. Tang, and Y. Chen, “A forward-imaging needle-type OCT probe for image guided stereotactic procedures,” Opt. Express 19, 26283-26294 (2011).
38. E. J. Min, J. Na, S. Y. Ryu, and B. H. Lee, “Single-body lensed-fiber scanning probe actuated by magnetic force for optical imaging,” Opt. Lett. 34, 1897-1899 (2009).

What is claimed is:

1. A system for obtaining information associated with at least one tissue, comprising:
at least one waveguide first arrangement which provides at least one first radiation to the at least one tissue, and which receives at least one optical second radiation from the at least one tissue;
at least one configuration which transceives at least one electrical signal to and from at least one portion of the system; and
at least one computing second arrangement which is configured to obtain the information based on the second radiation and data corresponding to the at least one electrical signal, wherein the data comprises a position of the at least one portion.

2. The system according to claim 1, wherein the configuration comprises an electrically-transmitting coating or material which transceives the at least one electrical signal.

3. The system according to claim 2, wherein the coating or the material at least partially covers the first arrangement.

4. The system according to claim 2, wherein the configuration further comprises a tube arrangement which is configured to transceiver at least one electrical further signal to and from the at least one portion of the system, wherein the coating or the material and the tube arrangement are electrically separate from one another.

5. The system according to claim 4, wherein the second arrangement generates the information further based on the electrical further signal.

6. The system according to claim 1, wherein the configuration further comprises a tube arrangement which is configured to transceiver at least one electrical further signal to and from the at least one portion of the system.

7. The system according to claim 5, wherein the tube arrangement comprises a drive shaft arrangement.

8. The system according to claim 1, wherein the configuration comprises at least one electrically-conducting wire which transceives the at least one electrical signal.

9. The system according to claim 8, wherein the at least one electrically-conducting wire comprises a plurality of wires.

10. The system according to claim 1, further comprising at least one third arrangement which is configured to at least one of translate or rotate at least one of (i) the first arrangement or (ii) the configuration, wherein the second arrangement determines the position based on simultaneous detection of the at least one second radiation and the at least one electrical signal during the at least one of the rotation or the translation.

11. The system according to claim 10, wherein the second arrangement further determines a spatial orientation of the at least one portion based on the simultaneous detection during the at least one of the rotation or the translation.

12. The system according to claim 1, wherein the second arrangement generates at least one three-dimensional image of the at least one tissue based on the information.

13. The system according to claim 1, wherein the second arrangement generates the information further based on at least one third radiation provided from a reference.

14. A probe apparatus, comprising:
at least one first arrangement which has at least one portion that physically contacts or penetrates at least one section of a tissue, and which transceives at least one optical radiation to or from the at least one section; and
at least one second arrangement which is configured to remove the at least one section from the tissue.

15. The apparatus according to claim 14, wherein, in operation, the at least one portion is at least one of translated or rotated within or near the tissue.

16. The apparatus according to claim 14, wherein the first arrangement is a needle.

17. The apparatus according to claim 16, wherein the second arrangement is a further needle.

18. The apparatus according to claim 17, wherein the needle and the further needle are situated in the apparatus in a substantially parallel manner with respect to one another.

19. The apparatus according to claim 17, further comprising a control arrangement which is configured to actuate at least one of (i) the needle, (ii) the further needle, or (iii) the apparatus by at least one of rotating or translating the same.

20. The apparatus according to claim 17, wherein the control arrangement actuates the needle and the further needle independently from one another.

21. The apparatus according to claim 14, wherein the second arrangement comprises a grasping arrangement which is configured to grasp the at least one section, and move the tissue out of the apparatus.

22. The apparatus according to claim 21, wherein the grasping arrangement is provided in a parallel configuration to the first arrangement.

23. The apparatus according to claim 21, wherein the grasping arrangement at least partially encloses the first arrangement.

24. The apparatus according to claim 14, wherein at least one of the first arrangement or the second arrangement includes a hydrophilic coating.

25. A method for obtaining information associated with at least one tissue, comprising:
with at least one waveguide first arrangement, providing at least one first radiation to the at least one tissue, and receiving at least one optical second radiation from the at least one tissue;
transceiving at least one electrical signal to and from at least one portion of the system; and
with at least one computing second arrangement, obtaining the information based on the second radiation and data corresponding to the at least one electrical signal, wherein the data comprises a position of the at least one portion.

26. A method using a probe apparatus, comprising:
with at least one first arrangement, physically contacting or penetrates at least one section of a tissue, and transceiving at least one optical radiation to or from the at least one section; and
removing the at least one section from the tissue.
